(19) United States
(12) Patent Application Publication
(10) Pub. No.: US 2023/0125698 A1
(43) Pub. Date: Apr. 27, 2023

(54) APHERESIS COLUMN FOR TREATING RHEUMATOID ARTHRITIS

(71) Applicants: UNIVERSITÉ PAUL SABATIER TOULOUSE III, Toulouse (FR); CENTRE HOSPITALIER UNIVERSITAIRE DE TOULOUSE, Toulouse (FR); INSTITUT NATIONAL DE LA SANTE ET DE LA RECHERCHE MEDICALE, Paris (FR); ASSISTANCE PUBLIQUE-HOPITAUX DE MARSEILLE, Marseille (FR); UNIVERSITE D'AIX-MARSEILLE, Marseille (FR)

(72) Inventors: Guy Serre, TOULOUSE CEDEX 9 (FR); Géraldine Offer, TOULOUSE CEDEX 9 (FR); Marianne Chabod, TOULOUSE CEDEX 9 (FR); Mikael Pancarte, TOULOUSE CEDEX 9 (FR); Jean Roudier, Marseille (FR); Nathalie Balandraud, Marseille (FR)

(73) Assignees: UNIVERSITÉ PAUL SABATIER TOULOUSE III, Toulouse (FR); CENTRE HOSPITALIER UNIVERSITAIRE DE TOULOUSE, Toulouse (FR); INSTITUT NATIONAL DE LA SANTE ET DE LA RECHERCHE MEDICALE, Paris (FR); ASSISTANCE PUBLIQUE-HOPITAUX DE MARSEILLE, Marseille (FR); UNIVERSITE D'AIX-MARSEILLE, Marseille (FR)

(21) Appl. No.: 17/913,416

(22) PCT Filed: Mar. 25, 2021
(86) PCT No.: PCT/EP2021/057849

§ 371 Date: Sep. 21, 2022

(30) Foreign Application Priority Data
Mar. 26, 2020 (FR) FR2002992

(51) Int. Cl. A61M 1/34; A61M 1/36

(52) U.S. Cl. CPC A61M 1/3486; A61M 1/3679; A61M 2202/0417

(57) ABSTRACT

The present invention relates to an apheresis column loaded with a solid support comprising a composition comprising at least one peptide selected from the group consisting of: —the αI7I-I85cit peptide of amino acid sequence VDIDIKIX₁SCX₂GSCS (SEQ ID NO: 8) wherein X₁ and X₂ each represent a citmllyl residue, —the α62I-635Cit peptide of amino acid sequence X1GHAKSX2PVX3GIHTS (SEQ ID NO: 12) wherein X1, X2 and X3 each represent a citmllyl residue—the P60-74cit-NH2 peptide of amino acid sequence X1PAPPPISGGGYX2AX3 (SEQ ID NO: 15) wherein X1 and X2 each represent a citmllyl residue and X3 represents a citmllyl derivative with a carboxamide group and—the peptide, referred to as the Ac-a36-50crt peptide, having the amino acid sequence GPX1VVEX2HQSACKDS (SEQ ID NO: 6) wherein the residue G at the N-terminal is acetylated and wherein X1 and X2 each represent a citmllyl residue and/or the a36-50cit peptide of amino acid sequence GPX1VVEX2HQSACKDS (SEQ ID NO: 5) wherein X₁ and X₂ each represent a citmllyl residue.

### TECHNICAL FIELD OF THE INVENTION

The present invention relates to a composition comprising one or more citrullinated peptides, an apheresis column having a solid support comprising the citrullinated peptide composition and a method for treating an autoimmune disease with anti-citrullinated protein autoantibodies using the citrullinated peptide composition or the apheresis column comprising the composition.

### TECHNICAL BACKGROUND

The rheumatoid arthritis (RA) is the most common human autoimmune disease and the most common chronic inflammatory rheumatism. It affects 0.5 to 1% of the population in developed countries and is characterised by a chronic and destructive inflammation of the joints. It is accompanied by the production of anti-immunoglobulin antibodies, referred to as “rheumatoid factors”, as well as anti-citrullinated peptide/protein antibodies (ACPA). The ACPA often appear years before the beginning of the disease (Rantapää-Dahlqvist S, et al. Arthritis Rheum. 2003 October; 48(10):2741-9; Nielen M M, et al. Arthritis Rheum. 2004 February; 50(2):380-6) and are present in 70 to 80% of the established RA (De Rycke L, et al. Ann. Rheum. Dis. (2004 63:12, 1587-1593, 2004). Due to their high specificity and presence at the beginning of the disease, they have a high diagnostic value. Often associated with a more severe evolution, they have a relative prognostic value. Finally, they also have a predictive value, as they can predict the evolution of an undifferentiated arthritis to a RA. The ACPA can be detected by various ELISA tests (Enzyme-Linked ImmunoSorbent Assay: immunoenzymatic test on microtiter plate). The tests classically used in medical practice are commercial tests referred to as anti-CCP (anti-Cyclic citrullinated peptides). The test developed and used by the inventors is the AhFibA test (Anti-human Fibrinogen Antibodies), which detects the antibodies recognising citrullinated fibrinogen. The results of the AhFibA and anti-CCP tests are largely consistent but discordant in 5 to 10% of patients.

As soon as the disease is diagnosed, various more or less specific treatments are prescribed to reduce the risk of evolution to irreversible joint injuries.

After the corticosteroids, the immunosuppressive drugs have become the first-line treatment prescribed to the majority of patients. In forms resistant to these treatments, various biotherapies such as anti-TNF-alpha, anti-IL6R, anti-CD20 are instituted, sometimes successively. All of these treatments have to be continued chronically, are not always effective or are effective for only a limited time and, in addition, all have more or less serious side effects. Finally, in some patients, despite the implementation of this therapeutic arsenal, the disease resists and progresses.

Therefore, there is still a need to find new ways to treat the RA, that are still effective and do not cause disabling side effects for the patient.

In recent years, the role of ACPA in the initiation and maintenance of the arthritis, through the formation of immune complexes with the citrullinated fibrinogen present in the joints of the patients, as demonstrated by the inventors, has been widely recognised by the international community. These immune complexes induce, via various effector mechanisms, the secretion of pro-inflammatory cytokines, in particular TNF-alpha, which provoke and maintain the joint inflammation and the synthesis of the ACPA.

### SUMMARY OF THE INVENTION

The inventors have identified from the citrullinated human fibrin, citrullinated peptides, referred to as immunodominant, which are the preferential targets of the ACPA and therefore capable of binding to them specifically. The inventors came up with the idea of ridding the body of the patients of the pathogenic ACPA with the help of these peptides. The inventors found that by using a column in which given citrullinated peptides are immobilised, they achieved near 100% purification rates of ACPA from patient plasma. They then designed a new device for the treatment of autoimmune diseases with anti-citrullinated protein autoantibodies such as rheumatoid arthritis.

An object of the present invention therefore relates to an apheresis column loaded with a solid support comprising a composition which comprises at least one peptide selected from the group consisting of:
the peptide, referred to as α171-185_{Cit}, having the amino acid sequence VDIDIKIX₁SCX₂GSCS (SEQ ID NO: 8) wherein X₁ and X₂ each represent a citrullyl residue,
the peptide, referred to as α621-635_{Cit}, having the amino acid sequence X₁GHAKSX₂PVX₃GIHTS (SEQ ID NO: 12) wherein X₁, X₂ and X₃ each represent a citrullyl residue,
the peptide, referred to as β60-74_{Cit-NH2}, having the amino acid sequence X₁PAPPPISGGGYX₂AX₃ (SEQ ID NO: 15) wherein X₁ and X₂ each represent a citrullyl residue and X₃ represents a citrullyl derivative with a carboxamide group in place of the carboxyl group and
the peptide, referred to as _{Ac}-α36-50_{Cit}, having the amino acid sequence GPX₁WEX₂HQSACKDS (SEQ ID NO: 6) wherein the N-terminal G residue is acetylated and wherein X₁ and X₂ each represent a citrullyl residue and/or the peptide, referred to as α36-50_{Cit}, having the amino acid sequence GPX₁WEX₂HQSACKDS (SEQ ID NO: 5) wherein X₁ and X₂ each represent a citrullyl residue,

whose peptide or peptides are immobilised directly or indirectly on the support.

The present invention also relates to a composition comprising at least two peptides selected from the group consisting of:
the peptide, referred to as α171-185_{Cit}, having the amino acid sequence VDIDIKIX₁SCX₂GSCS (SEQ ID NO: 8) wherein X₁ and X₂ each represent a citrullyl residue,
the peptide, referred to as α621-635_{Cit}, having the amino acid sequence X₁GHAKSX₂PVX₃GIHTS (SEQ ID NO: 12) wherein X₁, X₂ and X₃ each represent a citrullyl residue,
the peptide, referred to as β60-74_{Cit-NH2}, having the amino acid sequence X₁PAPPPISGGGYX₂AX₃ (SEQ ID NO: 15) wherein X₁ and X₂ each represent a citrullyl residue and X₃ represents a citrullyl derivative with a carboxamide group in place of the carboxyl group and
the peptide, referred to as _{Ac}-α36-50_{Cit}, having the amino acid sequence GPX₁WEX₂HQSACKDS (SEQ ID NO: 6) wherein the N-terminal G residue is acetylated and wherein X₁ and X₂ each represent a citrullyl residue and/or the peptide, referred to as α36-50_{Cit}, having the amino acid sequence GPX₁WEX₂HQSACKDS (SEQ ID NO: 5) wherein X₁ and X₂ each represent a citrullyl residue.

The present invention also relates to the use of the composition according to the invention in the manufacture of an apheresis column, preferably an apheresis column for the treatment of an autoimmune disease with anti-citrullinated protein autoantibodies such as the rheumatoid arthritis.

The present invention also relates to a composition according to the invention for its use in the treatment or the prevention, preferably by apheresis, of an autoimmune disease with anti-citrullinated protein autoantibodies and in particular the rheumatoid arthritis, wherein the peptide or the peptides of the composition are immobilised on a solid support.

### DETAILED DESCRIPTION OF THE INVENTION

Composition

The present invention relates to a composition comprising at least one peptide selected from the group consisting of:
the peptide, referred to as α171-185_{Cit}, having the amino acid sequence VDIDIKIX₁SCX₂GSCS (SEQ ID NO: 8) wherein X₁ and X₂ each represent a citrullyl residue,
the peptide, referred to as α621-635_{Cit}, having the amino acid sequence X₁GHAKSX₂PVX₃GIHTS (SEQ ID NO: 12) wherein X₁, X₂ and X₃ each represent a citrullyl residue
the peptide, referred to as β60-74_{Cit-NH2}, having the amino acid sequence X₁PAPPPISGGGYX₂AX₃ (SEQ ID NO: 15) wherein X₁ and X₂ each represent a citrullyl residue and X₃ represents a citrullyl derivative with a carboxamide group in place of the carboxyl group and
the peptide, referred to as _{Ac}-α36-50Cit, having the amino acid sequence GPX₁WEX₂HQSACKDS (SEQ ID NO: 6) wherein the N-terminal G residue is acetylated and wherein X₁ and X₂ each represent a citrullyl residue and/or the peptide, referred to as α36-50_{Cit}, having the amino acid sequence GPX₁WEX₂HQSACKDS (SEQ ID NO: 5) wherein X₁ and X₂ each represent a citrullyl residue.

The different sequences are summarised in Table 1 below.

TABLE 1

| | | SEQ |
|---|---|---|
| Protein/ | | ID |
| peptide | Amino acid sequence | NO |
| Human | MFSMRIVCLVLSVVGTAWTADSGEGDFLAEGGGVR | 1 |
| fibrino- | GPRVVERHQSACKDSDWPFCSDEDWNYKCPSGCRM | |
| gen α- | KGLIDEVNQDFTNRINKLKNSLFEYQKNNKDSHSL | |
| chain | TTNIMEILRGDFSSANNRDNTYNRVSEDLRSRIEV | |
| (NP_ | LKRKVIEKVQHIQLLQKNVRAQLVDMKRLEVDIDI | |
| 068657) | KIRSCRGSCSRALAREVDLKDYEDQQKQLEQVIAK | |
| (AAI01936) | DLLPSRDRQHLPLIKMKPVPDLVPGNFKSQLQKVP | |
| | PEWKALTDMPQMRMELERPGGNEITRGGSTSYGTG | |
| | SETESPRNPSSAGSWNSGSSGPGSTGNRNPGSSGT | |
| | GGTATWKPGSSGPGSTGSWNSGSSGTGSTGNQNPG | |
| | SPRPGSTGTWNPGSSERGSAGHWTSESSVSGSTGQ | |
| | WHSESGSFRPDSPGSGNARPNNPDWGTFEEVSGNV | |
| | SPGTRREYHTEKLVTSKGDKELRTGKEKVTSGSTT | |
| | TTRRSCSKTVTKTVIGPDGHKEVTKEVVTSEDGSD | |
| | CPEAMDLGTLSGIGTLDGFRHRHPDEAAFFDTAST | |
| | GKTFPGFFSPMLGEFVSETESRGSESGIFTNTKES | |
| | SSHHPGIAEFPSRGKSSSYSKQFTSSTSYNRGDST | |
| | FESKSYKMADEAGSEADHEGTHSTKRGHAKSRPVR | |
| | GIHTSPLGKPSLSP | |
| Human | MKRMVSWSFHKLKTMKHLLLLLLCVFLVKSQGVND | 2 |
| fibrino- | NEEGFFSARGHRPLDKKREEAPSLRPAPPPISGGG | |
| gen β- | YRARPAKAAATQKKVERKAPDAGGCLHADPDLGVL | |
| chain | CPTGCQLQEALLQQERPIRNSVDELNNNVEAVSQT | |
| (AAA18024) | SSSSFQYMYLLKDLWQKRQKQVKDNENVVNEYSSE | |
| | LEKHQLYIDETVNSNIPTNLRVLRSILENLRSKIQ | |
| | KLESDVSAQMEYCRTPCTVSCNIPVVSGKECEEII | |
| | RKGGETSEMYLIQPDSSVKPYRVYCDMNTENGGWT | |
| | VIQNRQDGSVDFGRKWDPYKQGFGNVATNTDGKNY | |
| | CGLPGEYWLGNDKISQLTRMGPTELLIEMEDWKGD | |
| | KVKAHYGGFTVQNEANKYQISVNKYRGTAGNALMD | |
| | GASQLMGENRTMTIHNGMFFSTYDRDNDGWLTSDP | |
| | RKQCSKEDGGGWWYNRCHAANPNGRYYWGGQYTWD | |
| | MAKHGTDDGVVMNWKGSWYSMRKMSMKIRPFFPQQ | |
| α36-50 | GPRVVERHQSACKDS | 3 |
| _{Ac}-α36-50 | GPRVVERHQSACKDS wherein G in N- | 4 |
| | terminal is acetylated | |
| α36-50_{Cit} | GPX₁VVEX₂HQSACKDS wherein X₁ and X₂ | 5 |
| | each represent a citrullyl residue | |
| _{Ac}-α36- | GPX₁VVEX₂HQSACKDS wherein G in N- | 6 |
| 50Cit | terminal is acetylated and wherein | |
| | X₁ and X₂ each represent a | |
| | citrullyl residue, | |
| α171-185 | VDIDIKIRSCRGSCS | 7 |
| α171-185_{Cit} | VDIDIKIX₁SCX₂GSCS wherein X₁ and X₂ | 8 |
| | each represent a citrullyl residue | |
| α501-515 | SGIGTLDGFRHRHPD | 9 |
| α501-515_{Cit} | SGIGTLDGFX₁HX₂HPD wherein X₁ and X₂ | 10 |
| | each represent a citrullyl residue | |
| α621-635 | RGHAKSRPVRGIHTS | 11 |
| α621-635_{Cit} | X₁GHAKSX₂PVX₃GIHTS wherein X₁, X₂ | 12 |
| | and X₃ each represent a citrullyl | |
| | residue | |
| β60-74 | RPAPPPISGGGYRAR | 13 |
| β60-74Cit | X1PAPPPISGGGYX₂AX₃ wherein X₁, X₂ | 14 |
| | and X₃ each represent a citrullyl | |
| | residue | |
| β60-74Cit- | X₁PAPPPISGGGYX₂AX₃ wherein X₁ and | 15 |
| NH2 | X₂each represent a citrullyl | |
| | residue and X₃ represents a | |
| | citrullyl derivative with a | |
| | carboxamide group (CONH₂) | |
| | in place of the terminal | |
| | carboxyl group (COOH). | |

The composition according to the invention also includes derivatives or fragments of the peptides defined above. The derivatives of these peptides may, for example, carry modifications intended to facilitate their synthesis and/or improve their stability.

Examples of such derivatives are peptides including amino acids whose carboxyl groups are esterified or transformed to amide groups. In the case of the β60-74_{Cit-NH2} peptide, a derivative of the latter may be the peptide referred to as β60-74_{Cit} of amino acid sequences SEQ ID NO: 14 in which the C-terminal citrullyl residue is not amidated.

The derivatives of the peptides according to the invention may also comprise peptides including amino acids in which an amino group is for example methylated, homocitrullinated, carbamylated or acetylated.

An example is the _{Ac}-α36-50_{Cit} peptide which is an N-terminal acetylated derivative of the α36-50_{Cit} peptide. The peptides α36-50_{Cit} or _{Ac}-α36-50_{Cit} can be used as a variant of each other in the present invention. However, the _{Ac}-α36-50_{Cit} peptide is preferred.

The composition according to the invention may comprise at least 1, 2, 3, 4 or 5 peptides selected from the group consisting of α171-185_{Cit}, α621-635_{Cit}, β60-74_{Cit-NH2} and _{Ac}-α36-50Cit and/or α36-50_{Cit}, or derivatives or fragments thereof.

Preferably, the composition according to the invention may comprise at least 1, 2, 3 or 4 peptides selected from the group consisting of _{Ac}-α36-50Cit, α171-185_{Cit}, α621-635_{Cit} and β60-74_{Cit-NH2} or derivatives or fragments thereof.

The composition according to the invention may comprise 1, 2, 3, 4 or 5 peptides selected from the group consisting of α171-185_{Cit}, α621-635_{Cit} β60-74_{Cit-NH2} and α36-50_{Cit} and/or _{Ac}-α36-50_{Cit}, or derivatives or fragments thereof.

Preferably, the composition according to the invention comprises 1, 2, 3, 4 or 5 peptides selected from the group consisting of _{Ac}-α36-50Cit, α171-185_{Cit}, α621-635_{Cit} and β60-74_{Cit-NH2} or derivatives or fragments thereof.

More preferably, the composition according to the invention comprises the 4 peptides: _{Ac}-α36-50_{Cit}, α171-185_{Cit}, α621-635_{Cit} and β60-74_{Cit-NH2}.

Even more preferably, the composition according to the invention consists of _{Ac}-α36-50Cit, α171-185_{Cit}, α621-635_{Cit} and β60-74_{Cit-NH2}.

The composition according to the invention may also consist of 1, 2, 3 or 4 peptides selected from the group consisting of _{Ac}-α36-50Cit, α171-185_{Cit}, α621-635_{Cit} and β60-74_{Cit-NH2} or derivatives or fragments thereof.

The composition according to the invention may further comprise the peptide, referred to as α501-515_{Cit}, having the amino acid sequence SGIGTLDGFX₁HX₂HPD (SEQ ID NO: 10) wherein X₁ and X₂ each represent a citrullyl residue or derivatives or fragments thereof. Also, according to one embodiment, the composition comprises and/or consists of at least 1, 2, 3, 4, 5 or 6 peptides or 1, 2, 3, 4, 5 or 6 peptides selected from the group consisting of _{Ac}-α36-50_{Cit} and/or α36-50Cit, α171-185_{Cit}, α621-635_{Ci}, β60-74_{Cit-NH2} and α501-515_{Cit} or derivatives or fragments thereof.

The reactivity profile of the ACPA differs between patients. Thus the inventors have shown that most of the plasmas tested were reactive to the β60-74_{Cit-NH2} and/or _{Ac}-α36-50_{Cit} peptide or peptides.

Also, according to one embodiment, the composition according to the invention comprises at least the β60-74_{Cit-NH2} and/or _{Ac}-α36-50_{Cit} peptides (or alternatively α36-50_{Cit}).

However, there are plasmas that are not reactive to these peptides but are reactive to other peptides of the invention. It is therefore preferable to combine as many peptides as possible to cover the maximum number of types of reactivity profiles of the plasmas.

Preferably, the composition according to the invention therefore comprises at least the α171-185_{Cit}, α621-635_{Cit}, β60-74_{Cit-NH2} and _{Ac}-α36-50_{Cit} peptides (or alternatively α36-50_{Cit}). More preferably it comprises the α171-185_{Cit}, α621-635_{Cit} β60-74_{Cit-NH2} and _{Ac}-α36-50_{Cit} peptides (or alternatively α36-50_{Cit}) and optionally the α501-515_{Cit} peptide.

The present invention also relates to the use of the composition as defined above in the manufacture of an apheresis column, preferably an apheresis column for the treatment or prevention of any autoimmune disease with anti-citrullinated protein autoantibodies. The autoimmune disease with anti-citrullinated protein autoantibodies can be selected from the group consisting of the syndrome of Sjögren, juvenile idiopathic arthritis and rheumatoid arthritis. The autoimmune disease with anti-citrullinated protein autoantibodies is preferably the rheumatoid arthritis.

Apheresis Column

The present invention also relates to an apheresis column loaded with a solid support comprising the composition as defined above whose peptide or peptides are immobilised directly or indirectly on the support.

The term “loaded” means that the column carries or contains the solid support in such a way that a liquid such as blood or a blood product can flow through the column in contact with the solid support.

Generally speaking, the apheresis is a technique in which blood or a blood product from a subject is circulated ex-vivo (extracorporeally) through a medical device that modifies the latter by adding, removing and/or replacing component or components before reinjecting it into the subject. More specifically, plasmapheresis is when the product treated by apheresis is plasma. In particular, plasmapheresis has been described to desensitize ABO-incompatible kidney transplant candidates; these candidates had anti-ABO antibodies that compromised the tolerance of the ABO-incompatible graft (L. Rostaing et al., Treatment of large plasma volumes using specific immunoadsorption to desensitize ABO-incompatible kidney transplant candidates, J. Nephropathology. 2016; 5(3):90-97).

Thus, the apheresis column according to the invention allows the removal of the ACPA from the blood or a blood product such as the plasma of a subject.

In a conventional apheresis method, the blood is collected directly at the level of a vein or an artery of a subject. In some embodiments, the blood is separated into one or more blood products (e.g. a solid fraction comprising the red and white blood cells, platelets and a liquid fraction such as plasma). A component (e.g. autoantibodies) is removed from one of the blood products (e.g. plasma). Eventually, the blood products are combined. The blood can then be reinjected into the vein or the artery of the subject.

The present invention relates in particular to an apheresis column loaded with a solid support comprising at least 1, at least 2, at least 3, at least 4 or at least 5 or 1, 2, 3, 4 or 5 peptide or peptides selected from the group consisting of:
the peptide, referred to as α171-185_{Cit}, having the amino acid sequence VDIDIKIX₁SCX₂GSCS (SEQ ID NO: 8) wherein X₁ and X₂ each represent a citrullyl residue,
the peptide, referred to as α621-635_{Cit}, having the amino acid sequence X₁GHAKSX₂PVX₃GIHTS (SEQ ID NO: 12) wherein X₁, X₂ and X₃ each represent a citrullyl residue
the peptide, referred to as β60-74_{Cit-NH2}, having the amino acid sequence X₁PAPPPISGGGYX₂AX₃ (SEQ ID NO: 15) wherein X₁ and X₂ each represent a citrullyl residue and X₃ represents a citrullyl derivative with a carboxamide group in place of the carboxyl group and
the peptide, referred to as _{Ac}-α36-50Cit, having the amino acid sequence GPX₁WEX₂HQSACKDS (SEQ ID NO: 6) wherein the N-terminal G residue is acetylated and wherein X₁ and X₂ each represent a citrullyl residue and/or the peptide, referred to as α36-50_{Cit}, having the amino acid sequence GPX₁WEX₂HQSACKDS (SEQ ID NO: 5) wherein X₁ and X₂ each represent a citrullyl residue,

or derivatives or fragments thereof,

whose peptide or peptides are immobilised directly or indirectly on the support.

The solid support of the apheresis column may also comprise the peptide, referred to as α501-515_{Cit}, having the amino acid sequence SGIGTLDGFX₁HX₂HPD (SEQ ID NO: 10) wherein X₁ and X₂ each represent a citrullyl residue or derivatives or fragments thereof.

More preferably, the solid support of the apheresis column comprises the 4 peptides: _{Ac}-36-50Cit, α171-185_{Cit}, α621-635_{Cit} and β60-74_{Cit-NH2}.

The present invention also relates to an assembly of columns comprising at least 1, at least 2, at least 3, at least 4 or at least 5 or 1, 2, 3, 4 or 5 apheresis columns, each apheresis column loaded with a solid support comprising a peptide selected from the group consisting of:
the peptide, referred to as α171-185_{Cit}, having the amino acid sequence VDIDIKIX₁SCX₂GSCS (SEQ ID NO: 8) wherein X₁ and X₂ each represent a citrullyl residue,
the peptide, referred to as α621-635_{Cit}, having the amino acid sequence X₁GHAKSX₂PVX₃GIHTS (SEQ ID NO: 12) wherein X₁, X₂ and X₃ each represent a citrullyl residue
the peptide, referred to as β60-74_{Cit-NH2}, having the amino acid sequence X₁PAPPPISGGGYX₂AX₃ (SEQ ID NO: 15) wherein X₁ and X₂ each represent a citrullyl residue and X₃ represents a citrullyl derivative with a carboxamide group in place of the carboxyl group and
the peptide, referred to as _{Ac}-α36-50Cit, having the amino acid sequence GPX₁WEX₂HQSACKDS (SEQ ID NO: 6) wherein the N-terminal G residue is acetylated and wherein X₁ and X₂ each represent a citrullyl residue and/or the peptide, referred to as α36-50_{Cit}, having the amino acid sequence GPX₁WEX₂HQSACKDS (SEQ ID NO: 5) wherein X₁ and X₂ each represent a citrullyl residue

or derivatives or fragments thereof,

whose peptide or peptides are immobilised directly or indirectly on the support.

According to an embodiment, the assembly of apheresis column comprises 4 apheresis columns, each loaded with a solid support comprising the _{Ac}-α36-50_{Cit} peptide, the α171-185_{Cit} peptide, the α621-635_{Cit} peptide and the β60-74_{Cit-NH2} peptide respectively. According to an embodiment, the assembly of apheresis column may further comprise the α501-515_{Cit} peptide.

The peptides according to the invention are preferably synthetic peptides.

The peptides according to the invention can be biotinylated by methods known in the prior art. In specific embodiments, the peptide or peptides are biotinylated via a spacer group. The spacer allows the citrullinated peptide to be moved away from the solid support and facilitates its recognition by the ACPA. Any suitable spacer that allows the binding properties of the peptide according to the invention to the ACPA to be maintained can be used in the invention. In specific embodiments, the spacer is a polyethylene glycol (PEG) or an amino hexanoic acid (AHX).

The solid support for immobilising the peptides of the invention is known in the prior art. The solid support is preferably made of a material that does not activate blood cells. It is preferable to use a support treated with an anticoagulant agent, for example a heparinised support. Alternatively, the blood of the patient may be treated with an anticoagulant such as heparin prior to application to the support.

The solid support may be made of polymers such as polysaccharides, preferably of high molecular weight e.g. 100 kDa or more, such as agarose or cellulose. The polysaccharide of the support may be cross-linked or non-cross-linked. Other polymers such as carboxylated polystyrene can be used. The solid supports can be in the form of magnetic beads or glass.

The solid support can be porous or non-porous. It may be in the form of particles which may be spherical or irregular. The average particle size can range from 10 μm to 2 mm, preferably from 30 μm to 100 μm.

The support can be treated with an anticoagulant agent such as heparin.

Methods for immobilising peptides on a solid support are known. Thus a peptide according to the invention can be immobilised on the support directly or indirectly.

Advantageously, the peptide or the peptides selected from the group consisting of α171-185_{Cit}, α621-635_{Cit} and β60-74_{Cit-NH2}, and optionally the α501-515_{Cit} peptide, are immobilised directly or indirectly on the solid support by their N-terminal end and/or
the _{Ac}-α36-50Cit and/or α36-50_{Cit} peptide or peptides are immobilised directly or indirectly to the solid support by their C-terminal end.

Preferably, the peptides in α171-185_{Cit}, α621-635_{Cit} and β60-74_{Cit-NH2}, and optionally the α501-515_{Cit} peptide, are immobilised directly or indirectly on the solid support by their N-terminal end and the _{Ac}-α36-50_{Cit} peptide (or alternatively α36-50_{Cit}) is immobilised directly or indirectly on the solid support through its C-terminal end.

The indirect immobilisation can be made by means of a suitable intermediate compound. A preferred method for indirectly immobilising a peptide is based on the interaction between the biotin and an intermediate compound such as the avidin or the streptavidin. Thus, the biotinylating of the peptide and the use of the avidin or the streptavidin immobilised on the solid support allows a reliable attachment of the peptides to the solid support. Specifically, the method may comprise providing the peptide according to the invention in biotinylated form, providing a solid support having streptavidin (or alternatively avidin) immobilised on its surface, contacting the support with an aqueous solution of biotinylated peptide or peptides according to the invention and rinsing the support with an aqueous solvent.

Also, in a preferred embodiment, the solid support then comprises the streptavidin or the avidin immobilised directly thereon. The _{Ac}-α36-50_{Cit} peptide or peptides (or alternatively α36-50_{Cit}), α171-185_{Cit}, α621-635_{Cit} and β60-74_{Cit-NH2}, and optionally the α501-515_{Cit} peptide, are biotinylated and linked to the streptavidin (or the avidin). The citrullinated peptides can be biotinylated in C-terminal or in N-terminal. Preferably, the α171-185_{Cit}, α621-635_{Cit} and β60-74_{Cit-NH2} peptides, and optionally the α501-515Cit peptide, are biotinylated in N-terminal and the _{Ac}-α36-50_{Cit} peptide (or alternatively α36-50_{Cit}) is biotinylated in C-terminal. The _{Ac}-α36-50_{Cit} peptides (or alternatively α36-50_{Cit}), α171-185_{Cit}, α621-635_{Cit} and β60-74_{Cit-NH2}, and optionally the α501-515_{Cit} peptide, may be biotinylated via a spacer group, for example amino hexanoic acid (AHX) or alternatively PEG. In addition to the affinity between the biotin and the avidin or the streptavidin, the antibody-antigen interactions can also be used for the indirect immobilisation of peptides on a support.

Alternatively, the peptides can be immobilised directly onto a solid support using bio-conjugation techniques such as, for example, the direct immobilisation of peptides onto solid supports activated with cyanogen bromide via amino functionalities in the primary sequence of the peptide or the direct immobilisation of peptides onto solid supports containing activated epoxides via carboxyl functionalities in the primary sequence of the peptide. Alternatively, the “Click” chemistry can be used to immobilise peptides on solid supports, the peptide and the support being modified with the appropriate mutually reactive chemical functionalities (azides and alkynes). In other embodiments, the Staudinger ligation chemistry can be used to immobilise suitably modified peptides on the suitably derivatives solid supports.

Treatment Method

The present invention also relates to a composition as hereinbefore defined for use in an apheresis column, preferably an apheresis column for the treatment or the prevention of an autoimmune disease with anti-citrullinated protein autoantibodies.

Autoimmune disease with anti-citrullinated protein autoantibodies is a disease in which the body of a person develops antibodies against citrullinated proteins in that same person. Autoimmune disease with anti-citrullinated protein autoantibodies can be selected in particular from the group consisting of the syndrome of Sjögren, juvenile idiopathic arthritis and rheumatoid arthritis. Preferably, the autoimmune disease with anti-citrullinated protein autoantibodies is rheumatoid arthritis.

The present invention also relates to a composition as hereinbefore defined for use in the treatment or the prevention, preferably by apheresis, of an autoimmune disease with anti-citrullinated protein autoantibodies, preferably rheumatoid arthritis, wherein the peptide or the peptides of the composition are immobilised on a solid support.

Preferably, the solid support is loaded onto an apheresis column.

Preferably, the treatment or the prevention of an autoimmune disease with anti-citrullinated protein autoantibodies comprises treating in vitro the blood of a patient suffering from said autoimmune disease with an apheresis column loaded with a solid support comprising the composition as hereinbefore defined.

The present invention also relates to a composition as hereinbefore defined for use in an apheresis column, preferably an apheresis column for the treatment of rheumatoid arthritis.

The present invention also relates to the use of an apheresis column as defined above, in particular to the use of this apheresis column in the treatment or the prevention of an autoimmune disease with anti-citrullinated protein autoantibodies and more preferably rheumatoid arthritis.

The present invention also relates to a method for treatment or prevention by apheresis, in a subject requiring it in which an apheresis column as defined above is used.

The present invention also relates to a method for purifying anti-citrullinated peptide antibodies (ACPA) from the blood or a blood product of a subject, comprising:
the provision of an apheresis column as defined above
contacting this column with the blood or a blood product of the subject in such a way as to deplete the blood or the blood product of the subject from ACPA.

The subject of the invention is preferably a mammal, more preferably a human being.

In a preferred embodiment, the subject treated according to the invention is a subject with a severe form of rheumatoid arthritis. The subject may also be a subject with high titer of anti-citrullinated protein/peptide antibodies (ACPA). High titer ACPA subjects are defined as subjects with an OD≥0.9 in a dosing of the ACPA by an AhFibA test. An OD≥20.9 in AhFibA test corresponds to the 40% of rheumatoid arthritis patients with the highest rates of ACPA (FIG. 1).

The invention will be further illustrated by the following figures and examples. However, these examples and figures should not be construed as limiting the scope of the present invention.

### BRIEF DESCRIPTION OF THE FIGURES

FIG. 1 Distribution of the AhFibA titers determined on serum samples from 202 AhFibA-positive rheumatoid arthritis patients. The threshold values of Optical Density (the corrected OD defines the Titer) separating the quartiles are noted.

FIG. 2 Percentage of serums reactive in ELISA with respect to the 4 β60-74_{Cit-NH2}, _{Ac}-α36-50_{Cit}, α621-635_{Cit} and α171-185_{Cit} peptides, among 202 serums from AhFibA-positive rheumatoid arthritis patients.

FIG. 3 Distribution of 202 AhFibA-positive serums from rheumatoid arthritis patients according to their reactivity profile with respect to the 4 β60-74_{Cit-NH2}, _{Ac}-α36-50_{Cit}, α621-635_{Cit} and α171-185_{Cit} peptides.

FIG. 4 Correlations between the titers of the antibodies against the 4 β60-74_{Cit-NH2}, _{Ac}-α36-50_{Cit}, α621-635_{Cit} and α171-185_{Cit} peptides, for the 202 serums tested. At each intersection the Spearman coefficient and (p) of the correlation are noted.

FIG. 5 Two types of chromatography were used to simultaneously purify the anti-β60-74_{Cit-NH2}, anti-_{Ac}-α36-50_{Cit}, anti-α621-635_{Cit} and anti-α171-185_{Cit} antibodies: either 4 mono-peptide columns connected in series (A), or a single column containing a balanced mixture of the 4 mono-peptide matrices (B).

FIG. 6 Chromatographs made on pools of serums from RA patients, either on the 4 serially mounted columns, loaded with the β60-74_{Cit-NH2}, _{Ac}-α36-50_{Cit}, α621-635_{Cit} and α171-185_{Cit} peptides respectively (left histograms), or on a column containing the mixture of the 4 peptides (right histograms). The pool 97 consisted of 97 serum samples of the same volume from patients with increasing ACPA titers representative of those found in a RA patient population; the Pool 27 consisted of 27 serum samples of the same volume from patients with high ACPA titers (OD≥1.5). The pools (starting serum: SD—black bar), the non-retained fractions (FNR—white bar) and the eluates (grey bar), were analysed in AhFibA ELISA (n≥24), the results are expressed as corrected OD.

FIG. 7 Method for calculating the purification rate of a serum after chromatography. The non-retained fraction is analysed in AhFibA ELISA and the OD obtained is compared to those resulting from the progressive dilution of the starting serum (reference curve). The ratio of dilutions allows to determine the purification rate. In this example, 1/50 vs 1/400: 8 times less antibodies in the FNR, i.e. 12.5% of the antibodies present in the starting serum. 87.5% of the ACPA were therefore purified.

FIG. 8 Calculation of the ACPA purification rate of a serum (n°41) after chromatography on the 4 columns loaded respectively with the β60-74_{Cit-NH2}, _{Ac}-α36-50_{Cit}, α621-635_{Cit}, α171-185_{Cit} peptides mounted in series. The non-retained fraction was analysed in AhFibA ELISA and the OD obtained compared to those resulting from the dilution of the starting serum (reference curve). The ratio of 1/50 to 1/1718 dilutions allows to determine the purification rate: 98% of the ACPA have been purified.

FIG. 9 Chromatography made from the Pool 97, the Pool 27 and 10 individual serums, from RA patients on the 4 columns loaded with β60-74_{Cit-NH2}, _{Ac}-α36-50_{Cit}, α621-635_{Cit} and α171-185_{Cit} peptides respectively and mounted in series. For each pool and individual serum, the starting serums (SD—black bars) and the non-retained fractions (FNR—white bars) were analysed in AhFibA ELISA (n≥24): their titers are expressed as corrected OD. The purification rates (%) were calculated for all the chromatographed samples (grey bars).

FIG. 10 Correlation between the titer in ACPA (AhFibA ELISA) of the starting serum (before chromatography) and the purification rate obtained after chromatography on the 4 columns in series, loaded respectively with the β60-74_{Cit-NH2}, _{Ac}-α36-50_{Cit}, α621-635_{Cit} and α171-185_{Cit} peptides. The correlation is highly significant (p=0.014): the higher the ACPA titer of the serums, the more efficient the purification.

### EXAMPLES

Materials and Methods

Serum Samples

Serum samples were obtained from 202 rheumatoid arthritis patients positive for the “AhFibA” ELISA test, which allows to detect and titer the ACPA using the citrullinated human fibrinogen as an immunosorbent.

Synthetic Peptides

Peptides corresponding to residues 36 to 50, 171 to 185 and 621 to 635 of the α-chain of the human fibrinogen (NP_068657—Isoform 2) and residues 60 to 74 of its β-chain (AAA18024) were synthesised.

The peptides were synthesised in their citrullinated form (by systematic substitution of all the arginyl residues by a citrullyl residue) or in their native non-citrullinated form (arginyl residues).

The β60-74 and β60-74_{Cit} peptides were synthesised with a carboxamide in place of the terminal carboxyl group to give the β60-74_{NH2} and β60-74_{Cit-NH2} peptides.

The α36-50, and α36-50_{Cit} peptides were synthesised with an acetyl on the N-terminal side to obtain the _{Ac}-α36-50_{and Ac}-α36-50_{Cit} peptides.

The peptides were biotinylated at the C-terminal of an amino hexanoyl spacer for the _{Ac}-α36-50Cit peptide and at the N-terminal of the same spacer, for the α171-185_{Cit}, α621-635_{Cit} and β60-74_{Cit-NH2} peptides.

Tests ELISA

The antigens or immunosorbents (citrullinated fibrinogen or citrullinated and non-citrullinated peptides), solubilised in PBS (1.5 mM KH₂PO4 SIGMA 795488; 7 mM K₂HPO₄ SIGMA P3786; 0.15M NaCl SIGMA 31434; pH 7.2) at concentrations of 5 and 10 μg/mL, are adsorbed in the microtiter plate wells (MAXISORP NUNC 2023-09) by incubation overnight at 4° C., or used in avidin-biotin system: avidin 5 μg/mL in PBS incubated overnight at 4° C. and then, after washing, biotinylated peptide at 10 μg/mL in PBS, incubated 1 h at 4° C. After blocking in PBS 2% BSA (Sigma A3059) for 1 hour at 4° C. and washing in PBS 0.1% Tween®20 (SIGMA P1379), the samples to be tested are deposited diluted 1:50 (1:100 in the Avidin-Biotin system) or at equivalent corrected dilutions, in PBS 2% BSA 2MNaCl. After washing, the secondary antibody (goat anti-human IgG Fc fragment coupled to horseradish peroxidase; Southern Biotech 2040-05) is incubated for 1 hour at 4° C. After washing, the revelation is performed with a solution of Ortho-Phenylene Diamine dihydrochloride (Sigma P2788), 0.03% H₂O₂ (Sigma H1009), in citrate/H₃PO₄ buffer pH5 (0.05 M citric acid Sigma C0759; 0.1 M Na2HPO4 Prolabo 28026292) for 5 minutes at room temperature, and then stopped with a solution of H₂SO₄ 6N (Sigma 07208). The optical density (OD) is read at 492 nm with a Multiskan Fc plate reader (ThermoScientific).

Conditions for Carrying ELISA Tests

TABLE 2

| | Concentration | | Secondary |
|---|---|---|---|
| | of the | Sample | antibody |
| | immunosorbent | dilution | dilution |
| AhFibA | 5 μg/mL | 1/50 | 1/15000 |
| Anti-β60-74 | 10 μg/mL | 1/50 | 1/2500 |
| Anti-α36-50 | 10 μg/mL | 1/50 | 1/2500 |
| Anti-α621-635 | 10 μg/mL | 1/50 | 1/2500 |
| Anti-α171-185biot | 10 μg/mL | 1/100 | 1/2500 |

Expression of the results of the ELISA tests: for the peptides, the variation in OD between the reactivity of the citrullinated peptide and the reactivity of the non-citrullinated peptide is taken into account, each reactivity being previously subtracted from its blank (OD in the absence of sample). This gives the following formula:

ΔDO=(DO_{cit}−DO_{blank cit})−(DO_{noncit}−DO_{blank noncit}) [Math 1]

Note that for the fibrinogen, the non-citrullinated form never shows any reactivity and therefore does not need to be evaluated. For the AhFibA test the OD value is therefore simply subtracted from its blank (OD in the absence of sample).

The ΔDO for the peptides and the OD for the citrullinated fibrinogen are considered to express the titer of the samples tested.

The inter-assay variations are corrected for by using a range of dilutions of a pool of patient serums tested in each microtiter plate. This reference pool, named P97, was made up of 97 samples of the same volume from 97 patients with a high ACPA titer. The OD values of this internal range are compared to that of reference values where each point in the range is the average of 30 determinations previously performed from this same pool. The least squares method then allows to obtain a correction factor which is applied to all the OD values obtained on the corresponding plate. This corrected OD corresponds to the titer of the sample.

$$corrected DO = \frac{\sum X * Y *}{\sum {X}^{2}} * DO \qquad [ Math 2 ]$$

With: X internal values, Y: reference values

Construction of Chromatography Columns

Four 1 mL HiTrap Streptavidin HP columns (GE; 29-0513-24) are independently loaded with each of the 4 biotinylated β60-74_{Cit-NH2}, _{Ac}-α36-50_{Cit}, α621-635_{Cit} and α171-185_{Cit} peptides. After equilibration of the column in PBS, a column volume (1 mL) of saturating solution of biotinylated peptides is injected at 0.2 mL/min, incubated for 15 min and then recovered from the column and lyophilised.

The amount of peptide remaining in the lyophilized fraction is determined by high performance liquid chromatography (UPLC); this amount is subtracted from the starting amount to estimate the amount of peptide bound in the column.

TABLE 3

| | Amount of |
|---|---|
| | peptide bound |
| Peptide | (μmol) |
| β60-74_{Cit-NH2} | 0.83 |
| Ac-α36-50_{Cit} | 1.1 |
| α621-635_{Cit} | 0.87 |
| α171-185_{Cit} | 1.7 |

Four columns were loaded with the respective amounts of peptides shown in the table above.

After use for the serial purification tests, the 4 matrices of the columns loaded with the β60-74_{Cit-NH2}, α36_{Ac}-50 _{Cit}, α621-635_{Cit} and α171-185_{Cit} peptides were recovered and mixed and then packed into an empty 10 mL column (GE, C10/10) with volume adapter (GE, AC10), to form a column loaded with the 4 mixed peptides of a final volume of 4 mL.

Purification

Two representative pools, P97 (previously described) and P27, a pool of 27 patient serums with an ACPA titer greater than 1.5, as well as individual serums selected on the basis of their reactivity to the 4 β60-74_{Cit-NH2}, α36_{Ac}-50 _{Cit}, α621-635_{Cit} and α171-185_{Cit} peptides were diluted to ¼ in PBS before chromatography.

After equilibration of the columns in PBS at a flow rate of 0.5 mL/min, a volume of 6 mL of pool or serum thus diluted was injected onto the 4 columns mounted in series in the following order: column loaded with β60-74_{Cit-NH2} peptide, then α36_{Ac}-50 _{Cit}, α621-635_{Cit} and α171-185_{Cit}, and subsequently onto the column containing the 4 peptides. Fractions not retained were recovered. After washing in PBS, the elution of the antibodies specific for the 4 peptides was made with a 0.2M pH3 glycine-HCl buffer (Invitrogen; 15527-013). The eluted fractions were recovered and brought to pH 7 with a 2M Tris solution (Euromedex; 77-86-1). Starting serums, non-retained fractions and eluates were then tested in ELISA after correction of the dilution factor induced by chromatography.

Determination of the Percentage or Rate of Purification

For each pool or serum treated, the percentage of purification was calculated from the residual immunoreactivity in ACPA (AhFibA ELISA test) of the purified, non-retained fractions and a range of dilutions of the corresponding pool or starting serum.

$$\% purification = ( 1 - \frac{Dilution SD corresponding to FNR OD}{FNR experimental dilution} ) * 100 \qquad [ Math 3 ]$$

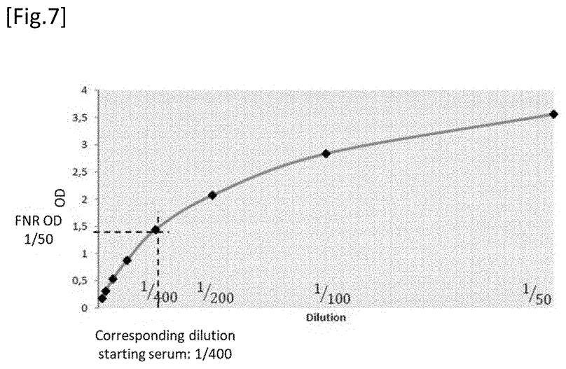

A fictitious example of the calculation of percentage purification is shown in FIG. 7 where to obtain an OD equivalent to that of the non-retained fraction (tested at 1/50 after correction for chromatographic dilution) the starting serum must be diluted to 1/400. The concentration of ACPA in the non-retained fraction is therefore 8 times lower than in the starting serum. One eighth, or 12.5%, of the ACPA in the starting serum are present in the non-retained fraction and have therefore not been purified. It can be deduced that seven-eighths of the ACPA in the starting serum, i.e. 87.5%, have been purified: this is the purification rate.

Reactivity Profile of the Serum Samples

The reactivity profiles of the 202 samples of patient serums with respect to the citrullinated fibrinogen and the 4 citrullinated peptides were determined by ELISA in at least two independent assays, each being done in duplicate. For the sake of reproducibility, only the microtiter plates whose internal range required the application of a correction factor between 0.5 and 2 with respect to the reference range were considered.

Results

ELISA Reactivity Profiles of Serums from Patients with Rheumatoid Arthritis (RA) with Respect to the Citrullinated Fibrinogen (AhFibA Test) and with Respect to the 4 Immunodominant Peptides.

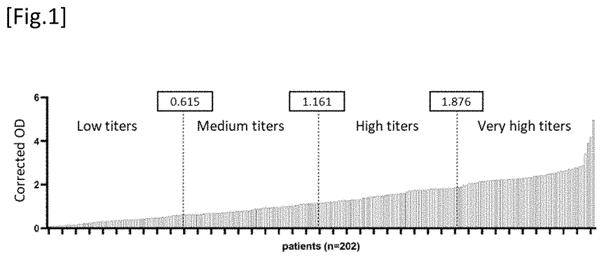

202 serums from RA patients were tested in AhFibA and ranked in order of increasing reactivity (OD or titer) (FIG. 1). According to their titer, the patients were divided into quartiles: low (OD=[0.056-0.615]), medium (OD=[0.615-1.161]), high (OD=[1.161-1.876]) and very high (OD≥1.876) titers.

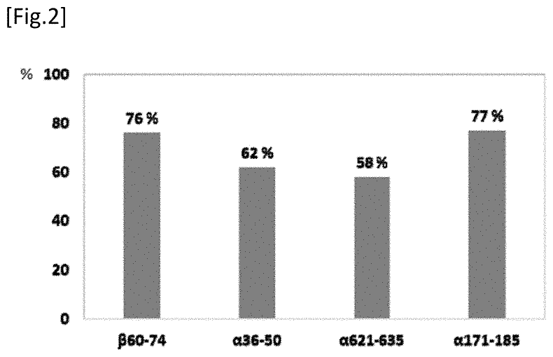

FIG. 2 shows the percentages of RA serums that are reactive with respect to each of the 4 peptides: 76% were positive for the β60-74Cit-NH2 peptide, confirming that it is the most immunodominant.

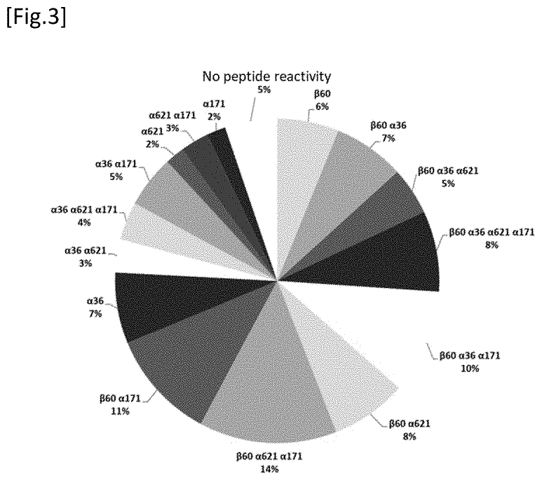

The individual reactivity of the serums with respect to the 4 peptides is highly variable and allows the definition of numerous reactivity profiles. The frequency of the different profiles observed is shown in FIG. 3. Among these 202 serums, 78% are reactive against at least 2 of the 4 peptides, and 95% react with at least one of the 4 peptides. These results show that the use of a combination of the 4 peptides in chromatography should allow to target a very broad spectrum of patients.

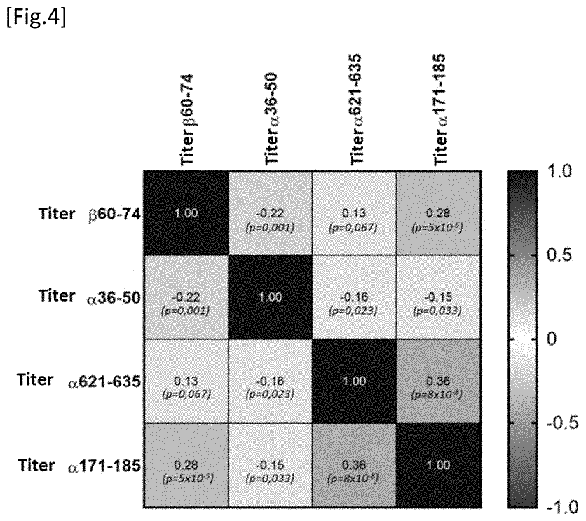

The search for correlations between the reactivities of the serums with respect to the different peptides reveals significant correlations between the β60-_{74Cit-NH2}, α621-635_{Cit} and α171-185_{Cit} peptides; these correlations reflect a certain degree of cross-reactivity between these peptides, but their relative weakness shows that each peptide has its own reactivity. However, no correlation was found between α36_{Ac}-50_{Cit} and the other 3 peptides. The double-entry table in FIG. 4 shows the Spearman coefficients calculated for all the peptide pairs compared and whether they are significant or not. These results support the idea that in order to develop a purification system targeting the whole of the ACPA for the largest possible number of patients, it is necessary to use all 4 peptides together. So in the end, this is the choice that is made.

Purification of Serum Pools from RA Patients on 4 Chromatography Columns Loaded with β60-74_{Cit-NH2}, _{Ac}-α36-50 _{Cit}, α621-635_{Cit} and α171-185_{Cit} Peptides Respectively and Mounted in Series, or on a Column Containing a Mixture of the 4 Peptides.

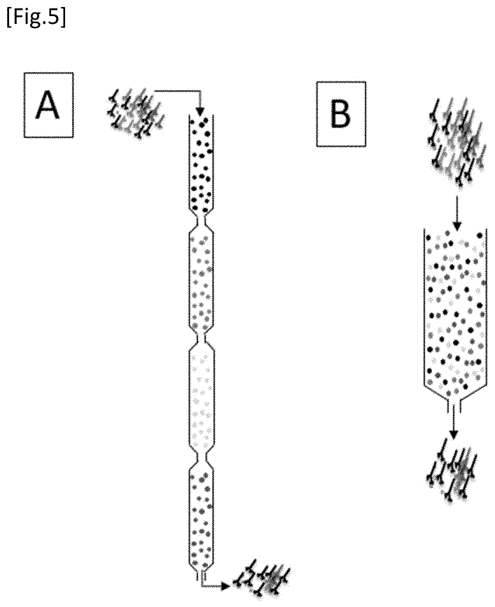

Two chromatography systems, shown in FIG. 5, were devised to purify the patient serums of the ACPA they contain using the 4 peptides. The first system consists of connecting in series 4 columns loaded respectively with one of the 4 β60-74_{Cit-NH2}, _{Ac}-α36-50_{Cit}, α621-635_{Cit} and α171-185_{Cit}(A) peptides, the second consists of mixing in equivalent quantities the 4 mono-peptide matrices and loading them into a single column (B).

The efficacy of the two systems was compared using two pools of serums from RA patients. These conditions were chosen because they are more demanding than those for testing an individual serum. Indeed, in a pool containing many serums with different reactivity profiles, the mixture of ACPA is highly polyclonal and composed of ACPA of different antigenic specificity.

A first pool, P97, consists of 97 serums with a distribution of the AhFibA titres representative of the general population of RA patients. A second pool, P27, consists of 27 serums from patients with high AhFibA titers (OD>1.5).

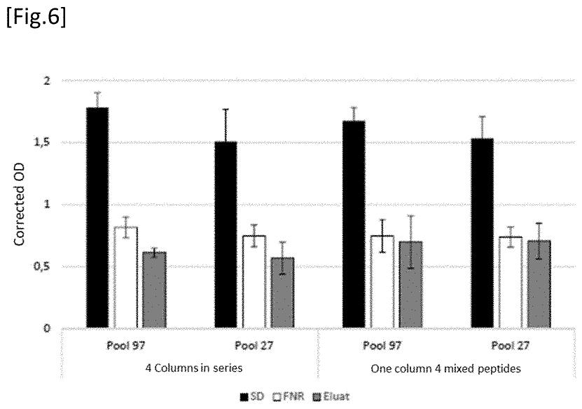

FIG. 6 shows the results of chromatography made on these pools, either on the 4 serially mounted columns, loaded with the β60-74_{Cit-NH2}, _{Ac}-α36-50_{Cit}, α621-635_{Cit} and α171-185_{Cit} peptides respectively, or on a column containing the mixture of the 4 peptides. A significant proportion of the ACPA has been absorbed onto the columns and can be eluted from them, while another portion, not absorbed, is found in the fraction not retained by the columns. It appears that both chromatography systems are effective in purifying a large portion of the ACPA contained in the pools and that they give little different results.

In addition, 10 ACPA-positive serums were selected based on their reactivity profiles with respect to the 4 β60-74_{Cit-NH2}, _{Ac}-α36-50_{Cit}, α621-635_{Cit} and α171-185_{Cit} peptides (Table 4 below).

TABLE 4

| Serum | | | | |
|---|---|---|---|---|
| no. | β60-74_{Cit-NH2} | _{Ac}-α36-50_{Cit} | α621-635_{Cit} | α171-185_{Cit} |
| n°1 | +++ | | + | + |
| n°10 | +++ | | | + |
| n°41 | | +++ | | ++ |
| n°16 | | + | +++ | ++ |
| n°29 | | + | +++ | |
| n°25 | ++ | +++ | + | |
| n°64 | +++ | +++ | | |
| n°22 | ++ | | ++ | +++ |
| n°6 | +++ | | +++ | + |
| n°17 | +++ | + | +++ | +++ |

(+++: strong positive; ++: medium positive; +: weak positive; empty box: negative)

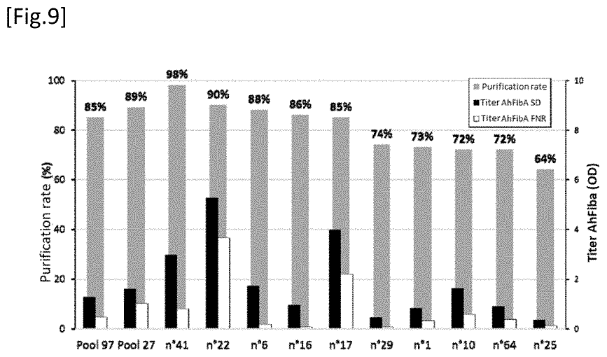

These serums as well as the two pools P97 and P27 were run on 4 serial columns loaded with the 4 β60-74_{Cit-NH2}, _{Ac}-α36-50_{Cit}, α621-635_{Cit} and α171-185_{Cit} peptides respectively. For all the samples, the ACPA titer decreased sharply after passing through the columns (FIG. 9).

An efficiency indicator of the purification was needed to compare the results between the different chromatographic methods and samples. As the optical density of a sample in ELISA is not linearly but exponentially correlated with the amount of ACPA contained in this sample, a simple ratio of optical densities between the titer of the starting serum and that of the fraction not retained on the columns could not allow to calculate a purification rate.

We therefore used a graphical method which consists of determining in the AhFibA ELISA the OD of a series of dilutions of the starting serum, allowing us to establish a reference curve. The OD of the non-retained fraction is determined in the same way, after correction for the chromatography-related dilution factor. The graphical reading on the reference curve indicates to which dilution of the starting serum the OD of the non-retained fraction corresponds. The percentage of purification calculated results from the ratio between the experimental dilution of the non-retained fraction and the corresponding dilution of the starting serum.

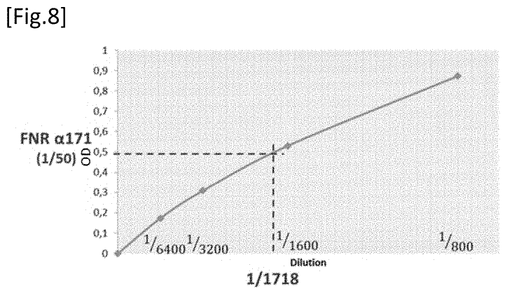

This graphical method of determining the purification rate is illustrated in FIG. 7. An example of the calculation of this rate for one of the treated serums (serum no. 41) is shown in FIG. 8.

The purification rates calculated for the 2 pools and for the 10 individual serums after a single pass through the columns are all above 65% and 7 out of 12, including the 2 pools, are above 85%. For the serum n° 41, this rate reaches 98%.

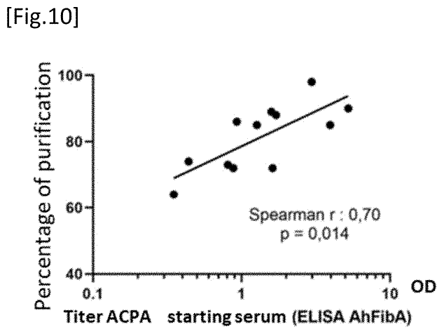

The relationship between the ACPA titer of the starting serum and the purification rate obtained after passage through the 4 columns mounted in series was studied. FIG. 10 shows that there is a strong and significant correlation (r of Spearman: 0.70; p=0.014) between these two parameters. The higher the titer in ACPA of the serum, the more efficient the purification. This counter-intuitive result is due to the fact that the high-titer ACPA have a high affinity and therefore bind more efficiently to their antigens.

### REFERENCES

Throughout this application, various references describe the prior art to which the invention belongs. The disclosures of these references are incorporated by reference into this application.

1. An apheresis column loaded with a solid support comprising a composition which comprises at least one peptide selected from the group consisting of:
a peptide, α171-185_{Cit}, comprising amino acid sequence VDIDIKIX₁SCX₂GSCS (SEQ ID NO: 8) wherein X₁ and X₂ each represent a citrullyl residue,
a peptide, α621-635_{Cit}, comprising amino acid sequence X₁GHAKSX₂PVX₃GIHTS (SEQ ID NO: 12) wherein X₁, X₂ and X₃ each represent a citrullyl residue,
a peptide, β60-74_{Cit-NH2}, comprising amino acid sequence X₁PAPPPISGGGYX₂AX₃ (SEQ ID NO: 15) wherein X₁ and X₂ each represent a citrullyl residue and X₃ represents a citrullyl derivative with a carboxamide group in place of the carboxyl group, and
a peptide, _{Ac}-α36-50_{Cit}, comprising amino acid sequence GPX₁VVEX₂HQSACKDS (SEQ ID NO: 6) wherein the N-terminal G residue is acetylated and wherein X₁ and X₂ each represent a citrullyl residue, and/or a peptide, α36-50_{Cit}, comprising amino acid sequence GPX₁VVEX₂HQSACKDS (SEQ ID NO: 5) wherein X₁ and X₂ each represent a citrullyl residue;
wherein the peptide or peptides are immobilised directly or indirectly on the solid support.

2. The apheresis column according to claim 1 wherein it comprises at least 2 peptides selected from the group consisting of α36-50_{Cit}, _{Ac}-α36-50_{Cit}, α171-185_{Cit}, α621-635_{Cit} and β60-74_{Cit-NH2}.

3. The apheresis column according to claim 1 comprising the α171-185_{Cit}, α621-635_{Cit}, β60-74_{Cit-NH2} peptides and the α36-50_{Cit} and/or the _{Ac}-α36-50_{Cit} peptide or peptides.

4. The apheresis column according to claim 1 further comprising α501-515_{Cit} peptide comprising amino acid sequence SGIGTLDGFX₁HX₂HPD (SEQ ID NO: 10) wherein X₁ and X₂ each represent a citrullyl residue.

5. The apheresis column according to claim 1 comprising:
the peptide or the peptides selected from the group consisting of α171-185_{Cit}, α621-635_{Cit}, and β60-74_{Cit-NH2}, and optionally the α501-515_{Cit} peptide, immobilised directly or indirectly on the solid support by their N-terminal end and/or
the _{Ac}-α36-50_{Cit} and/or α36-50_{Cit} peptide or peptides immobilised directly or indirectly on the solid support by their C-terminal end.

6. The apheresis column according to claim 1 that comprises the peptide or the peptides immobilised indirectly on the solid support by means of one or more intermediate compounds.

7. A composition comprising at least two peptides selected from the group consisting of:
a peptide, α171-185_{Cit}, comprising amino acid sequence VDIDIKIX₁SCX₂GSCS (SEQ ID NO: 8) wherein X₁ and X₂ each represent a citrullyl residue,
a peptide, α621-635_{Cit}, comprising amino acid sequence X₁GHAKSX₂PVX₃GIHTS (SEQ ID NO: 12) wherein X₁, X₂ and X₃ each represent a citrullyl residue,
a peptide, β60-74_{Cit-NH2}, comprising amino acid sequence X₁PAPPPISGGGYX₂AX₃ (SEQ ID NO: 15) wherein X₁ and X₂ each represent a citrullyl residue and X₃ represents a citrullyl derivative with a carboxamide group in place of the carboxyl group, and
a peptide, _{Ac}-α36-50_{Cit}, comprising amino acid sequence GPX₁VVEX₂HQSACKDS (SEQ ID NO: 6) wherein the N-terminal G residue is acetylated and wherein X₁ and X₂ each represent a citrullyl residue and/or
a peptide, α36-50_{Cit}, comprising amino acid sequence GPX₁VVEX₂HQSACKDS (SEQ ID NO: 5) wherein X₁ and X₂ each represent a citrullyl residue.

8. Manufacturing an apheresis column comprising linking the composition as defined in claim 1 to a solid support.

9. A method for the treatment or the prevention of an autoimmune disease; comprising the administration of anti-citrullinated protein autoantibodies according to claim 1, wherein the peptide or the peptides of the composition are immobilised on a solid support.

10. The method according to claim 9, wherein the autoimmune disease is selected from the group of Sjögren syndrome, juvenile idiopathic arthritis, and rheumatoid arthritis.

11. The method according to claim 9 wherein, the treated subject has a high titer of anti-citrullinated protein autoantibodies (ACPA).

12. The method according to claim 10, wherein the autoimmune disease is rheumatoid arthritis.

13. The apheresis column according to claim 2, comprising the α171-185_{Cit}, α621-635_{Cit}, and β60-74_{Cit-NH2} peptides and the α36-50_{Cit} and/or _{Ac}-α36-50_{Cit} peptide or peptides.


SEQUENCE LISTING (15 sequences)

SEQ ID NO: 1 - protein, 644 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 2 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 3 - protein, 15 aa, Artificial Sequence
  peptide
GPRVVERHQSACKDS

SEQ ID NO: 4 - protein, 15 aa, Artificial Sequence
  peptide
  MOD_RES (1)..(1): ACETYLATION
GPRVVERHQSACKDS

SEQ ID NO: 5 - protein, 15 aa, Artificial Sequence
  peptide
  MISC_FEATURE (3)..(3): X = citrullyl residue
  MISC_FEATURE (7)..(7): X = citrullyl residue
GPXVVEXHQSACKDS

SEQ ID NO: 6 - protein, 15 aa, Artificial Sequence
  peptide
  MOD_RES (1)..(1): ACETYLATION
  MISC_FEATURE (3)..(3): citrullyl residue
  MISC_FEATURE (7)..(7): citrullyl residue
GPXVVEXHQSACKDS

SEQ ID NO: 7 - protein, 15 aa, Artificial Sequence
  peptide
VDIDIKIRSCRGSCS

SEQ ID NO: 8 - protein, 15 aa, Artificial Sequence
  peptide
  MISC_FEATURE (8)..(8): X = citrullyl residue
  MISC_FEATURE (11)..(11): X = citrullyl residue
VDIDIKIXSCXGSCS

SEQ ID NO: 9 - protein, 15 aa, Artificial Sequence
  peptide
SGIGTLDGFRHRHPD

SEQ ID NO: 10 - protein, 15 aa, Artificial Sequence
  peptide
  MISC_FEATURE (10)..(10): X = citrullyl residue
  MISC_FEATURE (12)..(12): X = citrullyl residue
SGIGTLDGFXHXHPD

SEQ ID NO: 11 - protein, 15 aa, Artificial Sequence
  peptide
RGHAKSRPVRGIHTS

SEQ ID NO: 12 - protein, 15 aa, Artificial Sequence
  peptide
  MISC_FEATURE (1)..(1): X = citrullyl residue
  MISC_FEATURE (7)..(7): X = citrullyl residue
  MISC_FEATURE (10)..(10): X = citrullyl residue
XGHAKSXPVXGIHTS

SEQ ID NO: 13 - protein, 15 aa, Artificial Sequence
  peptide
RPAPPPISGGGYRAR

SEQ ID NO: 14 - protein, 15 aa, Artificial Sequence
  peptide
  MISC_FEATURE (1)..(1): X = citrullyl residue
  MISC_FEATURE (13)..(13): X = citrullyl residue
  MISC_FEATURE (15)..(15): X = citrullyl residue
XPAPPPISGGGYXAX

SEQ ID NO: 15 - protein, 15 aa, Artificial Sequence
  peptide
  MISC_FEATURE (1)..(1): X = rsidu citrullyl
  MISC_FEATURE (13)..(13): X = rsidu citrullyl
  MISC_FEATURE (15)..(15): X = rsidu citrullyl
  MOD_RES (15)..(15): AMIDATION
XPAPPPISGGGYXAX